(19) United States
(12) Patent Application Publication
(10) Pub. No.: US 2018/0338490 A1
(43) Pub. Date: Nov. 29, 2018

(54) APPARATUS, SYSTEMS, AND METHODS FOR PROCESSING PLATELETS AND CONTAMINATED BLOOD

(71) Applicants: Jimmy Walker, Arlington, MA (US); William Walter, San Clemente, CA (US)

(72) Inventors: Jimmy Walker, Arlington, MA (US); William Walter, San Clemente, CA (US)

(21) Appl. No.: 16/042,124

(22) Filed: Jul. 23, 2018

Related U.S. Application Data
(63) Continuation of application No. 14/200,549, filed on Mar. 7, 2014, now abandoned.
(60) Provisional application No. 61/774,927, filed on Mar. 8, 2013.

(51) Int. Cl. A01N 1/02; A61M 1/02; A61J 1/14; A61J 1/10; A61M 1/36

(52) U.S. Cl. CPC A01N 1/0268; A01N 1/0242; A61M 1/0281; A61M 1/0272; A61M 1/025; A61J 2200/42; A61J 1/1487; A61J 1/10; A01N 1/0284; A61M 1/36; A61J 2200/44; A01N 1/0263

(57) ABSTRACT

One aspect of the present disclosure relates to a bag for storing platelets or contaminated blood previously withdrawn from a subject. The bag can comprise a flexible, closed container and at least one removable heat transfer member adhered to an outer surface portion of the container. The container can include an inlet therein that forms part of the container. The inlet can be adapted for fluid-tight connection to a tube member through which the platelets or contaminated blood are caused to flow.

### RELATED APPLICATION

This application claims the benefit of U.S. Provisional Patent Application Ser. No. 61/774,927, filed Mar. 8, 2013, the entirety of which is hereby incorporated by reference for all purposes.

### TECHNICAL FIELD

The present disclosure relates generally to apparatus, systems, and methods for processing blood, and more particularly to apparatus, systems, and methods for freezing or thawing platelets and contaminated blood to increase the shelf life thereof.

### BACKGROUND

A wide variety of injuries and medical procedures require the transfusion of whole blood or a variety of blood components. Blood transfusions are routinely used to increase oxygen delivery capacity and circulatory volume in patients. Safe, quick, and easy access to transfusable blood cell units is not only important for trauma victims with massive blood loss, but also for patients undergoing elective surgery or who have diseases, such as several types of hereditary hemolytic anemias, which result in a loss of circulating red blood cells. The ability to store red blood cells for extended time periods ensures that a supply of transfuable red blood cells will be available when needed. Potential uses include storage of unique blood types, units intended for autologous transfusion and stockpiling general blood types for emergency situations. Limitations in the current storage methodologies have led, in part, to occasional shortages of blood supply, resulting in postponement of elective surgeries and calls for donations from blood banks and hospitals.

### SUMMARY

The present disclosure relates generally to apparatus, systems, and methods for processing blood, and more particularly to apparatus, systems, and methods for freezing or thawing platelets and contaminated blood to increase the shelf life thereof.

One aspect of the present disclosure relates to a bag for storing platelets or contaminated blood previously withdrawn from a subject. The bag can comprise a flexible, closed container and at least one removable heat transfer member adhered to an outer surface portion of the container. The container can include an inlet therein that forms part of the container. The inlet can be adapted for fluid-tight connection to a tube member through which the platelets or contaminated blood are caused to flow.

Another aspect of the present disclosure relates to a system for freezing or thawing platelets or contaminated blood previously withdrawn from a subject. The system can comprise an agitator drive, a linkage arm, a reciprocating arm, and a container. The linkage arm can be configured to translate a rotary motion generated by the agitator drive to an angular reciprocation motion. The reciprocating arm can be operably connected to the linkage arm. The container be configured to hold the platelets or contaminated blood. The container can also be operably connected to a distal end of the reciprocating arm. Operation of the system can provide a wrist-action motion to the container and thereby minimizes the amount of RBC or platelet lysis caused by freezing or thawing of the platelets or contaminated blood.

Another aspect of the present disclosure can include a method for freezing or thawing platelets or contaminated blood previously withdrawn from a subject. One step of the method can include providing a system comprising an agitator drive, a linkage arm operably connected to the agitator drive, a reciprocating arm operably connected to the linkage arm, and a container operably connected to a distal end of the reciprocating arm. Next, a portion of the container can be filled with the platelets or contaminated blood. A portion of the container can then be immersed in a cooling or warming fluid. The agitator drive can be activated to impart a wrist-action motion to the container for a time and at a speed sufficient to decrease the amount of RBC or platelet lysis resulting from freezing or thawing of the platelets or contaminated blood.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a perspective showing a bag for storing platelets or contaminated blood previously withdrawn from a subject constructed in accordance with one aspect of the present disclosure;

FIG. 2 is a photograph showing a container for holding platelets or contaminated blood during freezing or thawing constructed in accordance with another aspect of the present disclosure;

FIG. 3 is a schematic illustration showing a system for freezing or thawing platelets or contaminated blood previously withdrawn from a subject constructed in accordance with another aspect of the present disclosure;

FIG. 4 is a photograph showing one example of the system in FIG. 3; and

FIG. 5 is a process flow diagram illustrating a method for freezing or thawing platelets or contaminated blood previously withdrawn from a subject according to another aspect of the present disclosure.

### DETAILED DESCRIPTION

### Definitions

Unless defined otherwise, all technical and scientific terms used herein have the same meaning as is commonly understood by one of skill in the art to which the present disclosure pertains.

In the context of the present disclosure, the term “blood” can generally refer to whole blood or any fraction thereof, such as plasma or serum.

As used herein, the term “whole blood” can refer to a body fluid (technically a tissue) that is composed of blood cellular components suspended in plasma. Blood cellular components include red blood cells (RBCs), white blood cells (including both leukocytes and lymphocytes) and platelets (also called thrombocytes).

As used herein, the term “contaminated blood” can refer to blood, typically obtained from a blood donor, that has, or is suspected of having, at least one microorganism (e.g., virus, bacteria, etc.) therein. In some instances, a microorganism may be present within a blood component (e.g., intracellular), such as within a RBC or white blood cell. In other instances, a microorganism may be present in an extracellular fluid (e.g., plasma) comprising the blood.

As used herein, the term “subject” can refer to any warm-blooded organism including, but not limited to, humans, pigs, rats, mice, dogs, goats, sheep, horses, monkeys, apes, rabbits, cattle, etc.

As used herein, the terms “neutralize” or “neutralized” when used with reference to microorganisms can mean physical destruction of the microorganisms, eliminating or sufficiently diminishing a microbial activity (e.g., an infective or pathogenic activity or ability), and/or eliminating or sufficiently diminishing a toxin associated therewith.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

### Overview

The present disclosure relates generally to apparatus, systems, and methods for processing blood, and more particularly to apparatus, systems, and methods for freezing or thawing platelets and contaminated blood to increase the shelf life thereof. Conventional systems and methods for freezing and thawing blood yield blood or blood components (e.g., platelets) with a relatively short shelf life. For example, the average shelf life of the red cell portion of a blood donation prepared and stored by conventional systems and methods is about 42 days. Additionally, the average shelf life of platelets prepared and stored by conventional systems and methods is about 5 days. This means that the RBC and platelet portions of the collected blood needs to be disposed of after a relatively short period of time. Consequently, conventional systems and methods for freezing and thawing blood and blood components (e.g., platelets) produce considerable waste and costs. As described in more detail below, the present disclosure advantageously provides apparatus, systems, and methods that decrease the amount of RBC or platelet lysis associated with the freeze/thaw process, thereby significantly increasing the shelf life of the red cell portion or platelets of blood processed by the present disclosure.

### Apparatus

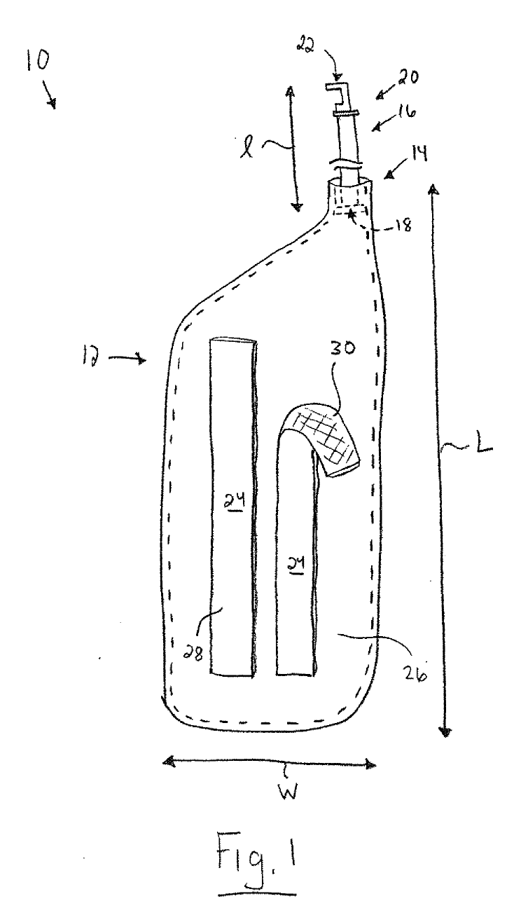

One aspect of the present disclosure can include a bag 10 (FIG. 1) for storing platelets or contaminated blood previously withdrawn from a subject. The bag 10 can comprise a flexible, closed container 12. The bag 10 can be biocompatible and made of one or more medical grade materials, such as a polymer or polymer blend. Non-limiting examples of materials that may be used to make the bag 10 can include PVC with citrate, di-2-ethylhexylphthalate (DEHP) or tri-2-ethylhexyl-tri-mellitate (TEHTM) plasticizer; polyolefin (PO), poly(ethylene-co-vinyl acetate) (EVA), or fluorinated polyethylene propylene (FEP). The bag 10 can have a regular or irregular shape. As shown in FIG. 1, for example, the bag 10 can have a generally rectangular shape. In one example, the bag 10 can have a length L of about 22 cm and a width W of about 19 cm.

The bag 10 can include at least one inlet 14 or port that forms part of the container 12. The inlet 14 can be adapted for fluid-tight connection to a tube member 16 through which platelets or contaminated blood can flow. Although only one inlet 14 is shown in FIG. 1, it will be appreciated that two, three, or more inlets can be included as part of the container 12. The inlet 14 can be configured to receive a distal end 18 of the tube member 16. In some instances, the inlet 14 can include a Luer fitting (not shown in detail) adapted to accommodate the distal end 18 of the member 16. In one example, the Luer fitting can be adapted to accommodate a tube member 16 having a 3/16internal diameter (ID). The tube member 16 can comprise an elongated, cylindrical member having a length 1 having a desired length 1. In one example, the length 1 of the tube member 16 can be about 30 mm. The proximal end 20 of the tube member 16 can include a connector 22 adapted to mate with any standard Luer fitting or 3/16ID tubing, for example. Examples of connectors 22 can include INTERLINK (Becton Dickinson Co., Franklin Lakes, N.J.), check valves, inline fittings, plugs and spike ports. The tube member 16 can be made any medical grade, biocompatible material(s) (e.g., PVC or FEP).

The bag 10 can additionally include at least one removable heat transfer member 24 adhered to an outer surface portion 26 of the container 12. Advantageously, the heat transfer members 24 optimize the heat transfer rate for maximum RBC or platelet recovery during and following freezing and thawing by allowing one to control the heat transfer rate (e.g., based on the number, size, and placement of the heat transfer members). In some instances, each of the heat transfer members 24 can be configured as a continuous, uninterrupted film or tape. As shown in FIG. 1, the bag 10 can include two heat transfer members 24; however, it will be appreciated that the bag can include any number of heat transfer members. The heat transfer members 24 can include a first side 28 and an oppositely disposed second side 30, which has a tacky surface (cross-hatched region) for adhering to a respective outer surface portion 26 of the container 12. Although the heat transfer members 24 are shown as having a rectangular shape, it will be appreciated that any other shape (e.g., circular, ovoid, square, etc.) is possible. Additionally, it will be appreciated that the heat transfer members 24 can be identically or differently shaped. The heat transfer members 24 can be made of any one or combination of materials that facilitate energy transfer between platelets or contaminated blood contained in the bag 10 and a cooling or warming fluid in which the bag is immersed. In one example, the heat transfer members 24 and the bag 10 are made of the same material(s). In another example, at least one heat transfer member 24 can be made of the same material(s) as the bag 10, while at least one heat transfer member is made of a material (or materials) different than the bag. The heat transfer members 24 can be easily removed (e.g., peeled) from the container 12 to optimize the heat transfer rate between its contents and the cooling or warming fluid.

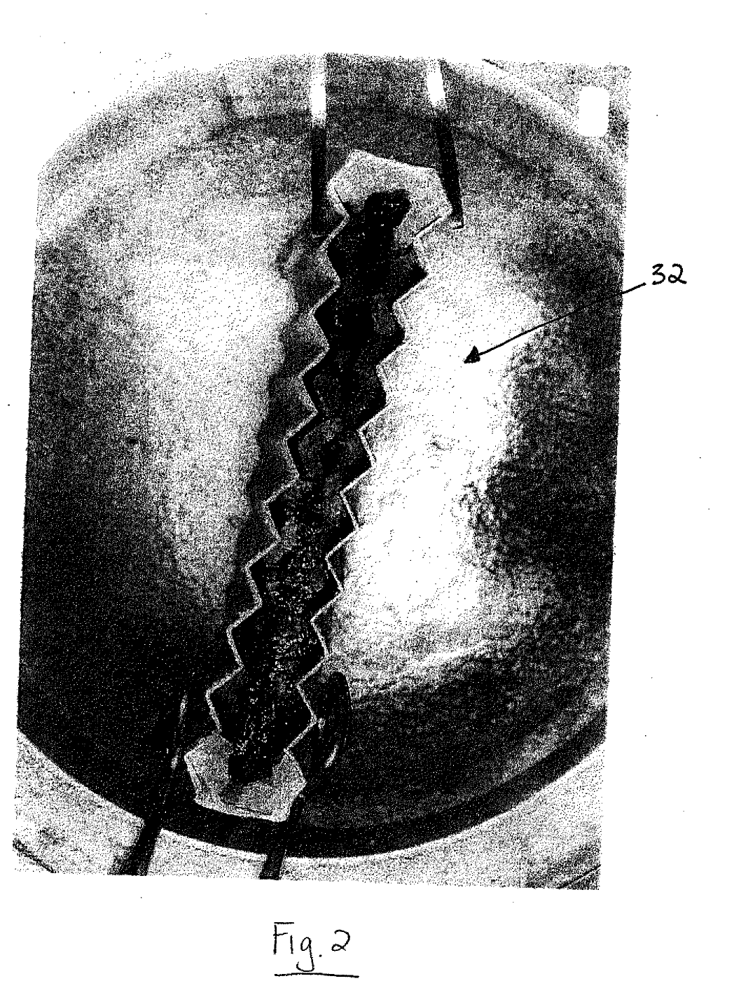

Another aspect of the present disclosure is shown in FIG. 2 and includes a sectioned corrugated container 32 configured to hold platelets or contaminated blood during freezing or thawing. The container 32 can be made of a conductive material, such as a metal (e.g., aluminum) The container 32 can have a generally rectangular shape and include a cavity for receiving platelets or contaminated blood. In one example, the cavity can be configured to hold a 250 ml mixture of platelets or contaminated blood and a cryoprotectant, such as polyvinylpyrrolidone (PVP). In some instances, the cryoprotectant glycerol may be substituted for PVP. Although the container 32 is shown in FIG. 2 as being removable from a cooling solution (e.g., using tongs), it will be appreciated that the container can include one or more attachment mechanisms (e.g., hooks, loops, etc.) (not shown) to facilitate handling of the container. Advantageously, the corrugated shape of the container 32 provides an increase surface-to-volume ratio (as compared to a container having planar or flattened sides) for optimal heat transfer.

### Systems

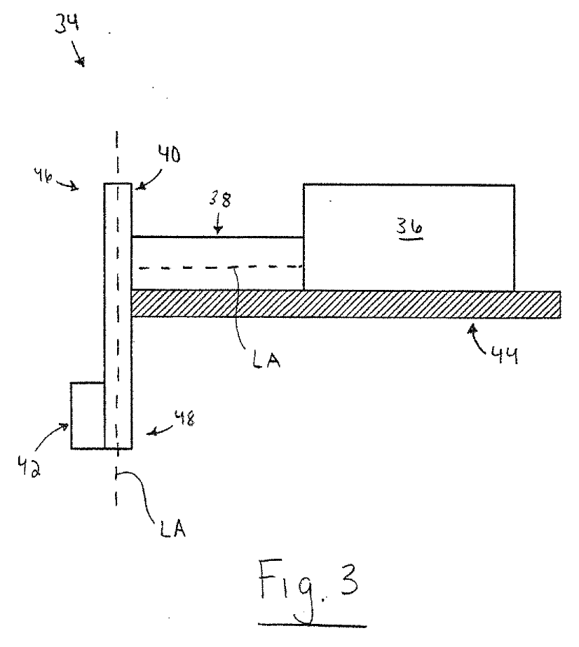

Another aspect of the present disclosure can include a system 34 (FIG. 3) for freezing or thawing platelets or contaminated blood previously withdrawn from a subject. The system 34 can generally comprise an agitator drive 36, a linkage arm 38, a reciprocating arm 40, and a container 42 configured to hold the blood. In some instances, the system 34 can be mounted on a substrate 44, such as a table. As described in more detail below, the system 34 advantageously provides a wrist-action that promotes heat transfer between the platelets or contaminated blood and the cooling or warming solution and thereby minimizes the time required to freeze or thaw the platelets or contaminated blood.

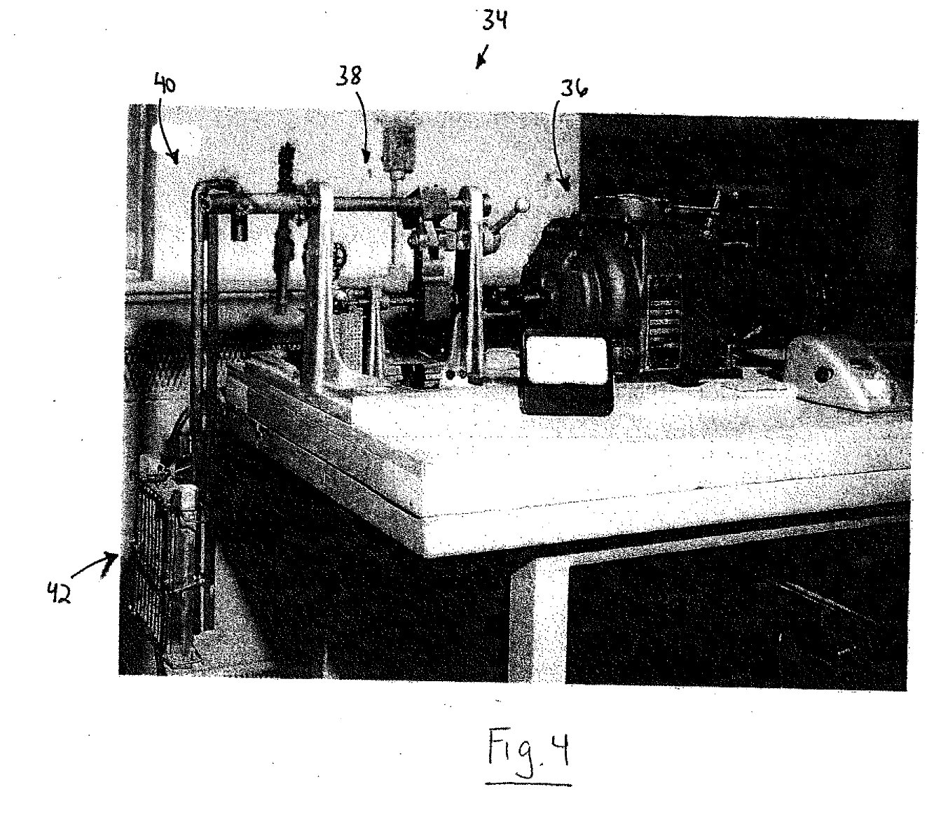

In some instances, the agitator drive 36 can comprise a variable speed drive motor capable of generating a rotary motion. The agitator drive 36 can be configured to adjust the angle and speed (rpm) of rotary motion. As described below, the angle and speed of the agitator drive 36 can be selectively adjusted to optimize the time required to freeze or thaw the platelets or contaminated blood. In one example, the agitator drive 36 can be configured to operate with a rotary angle of between about 80° and about 160° (e.g., about 120°). In another example, the agitator drive 36 can be configured to operate at a speed of about 30 rpm to about 80 rpm (e.g., about 50 rpm). In a further example, the agitator drive 36 can be configured to operate with a rotary angle of 120° and at a speed of 50 rpm. One example of an agitator drive 36 is shown in FIG. 4.

The linkage arm 38 can be operably connected (e.g., directly connected) to the agitator drive 36. The linkage arm 38 can be configured to translate the rotary motion generated by the agitator drive 36 to an angular reciprocation motion. In some instances, the linkage arm 38 can be made from plain carbon steel or aluminum The linkage arm 38 can additionally include standard bearings (not shown) to join or connect two or more segments thereof. One example of the linkage arm 38 is shown in FIG. 4.

The reciprocating arm 40 can be operably connected (e.g., directly connected) to the linkage arm 38. The reciprocating arm 40 can include a proximal end 46, an oppositely disposed distal end 48, and a longitudinal axis LA that extends between the proximal and distal ends. The longitudinal axis LA of the reciprocating arm 40 can extend substantially perpendicular to a longitudinal axis LA of the linkage arm 38. The reciprocating arm 40 can be made of one or more materials capable of withstanding a temperature range from about room temperature to about −320° F. One example of the reciprocating arm 40 is shown in FIG. 4.

The container 42 can be configured to hold the blood. Examples of containers are shown in FIGS. 1-2 and described above. The container 42 can be operably connected (e.g., directly connected) to the distal end 48 of the reciprocating arm 40. In one example, the corrugated metal container 32 can be operably connected to the distal end 48 of the reciprocating arm 40. In another example, the bag 10 can be similarly connected to the distal end 48 of the reciprocating arm 40.

As described in more detail below, operation of the systemsystecrc 34 provides a wrist-action motion to the container 42 (e.g., the bag 10 or the corrugated container 32). The wrist-action motion imparted to the container 42 (e.g., the bag 10 or the corrugated container 32) can be maintained for a time and at a speed sufficient to decrease the amount of RBC or platelet lysis caused by freezing or thawing of the platelets or contaminated blood. Additionally, the wrist-action motion imparted to the container 42 (e.g., the bag 10 or the corrugated container 32) minimizes the time required to freeze or thaw the platelets or contaminated blood.

### Methods

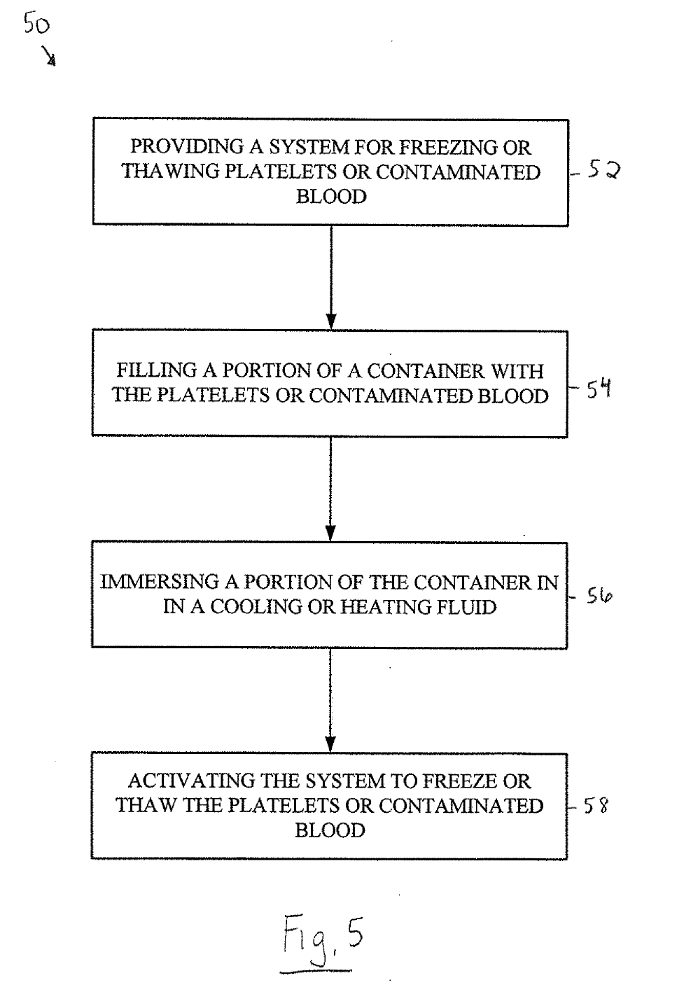

Another aspect of the present disclosure can include a method 50 (FIG. 5) for thawing or freezing platelets or contaminated blood previously withdrawn from a subject. As shown in FIG. 5, the method 50 can generally include the steps of: providing a system 34 that includes an agitator drive 36, a linkage arm 38 operably connected to the agitator drive, a reciprocating arm 40 operably connected to the linkage arm, and a container 42 operably connected to a distal end 48 of the reciprocating arm (Step 52); filling a portion of the container with the platelets or contaminated blood (Step 54); immersing a portion of the container in a cooling or warming fluid (Step 56); and activating the agitator drive to impart wrist-action motion to the container for a time and at a speed sufficient to decrease the amount of RBC or platelet lysis caused by freezing or thawing of the platelets or contaminated blood (Step 58).

At Step 52, a system 34, such as the one illustrated in FIGS. 3-4 and described above can be provided. Additional or optional system 34 components, such as the bag 10 or corrugated container 32 also discussed above, can be provided at Step 52.

At Step 54, a portion of the container 42 can be filled with a mixture of platelets or contaminated blood and cryoprotectant (e.g., glycerol). The platelets or contaminated blood can be obtained from a blood donor, for example. Thus, in some instances, the platelets or contaminated blood can be obtained as part of a whole blood sample withdrawn from a blood donor. In other instances, the whole blood sample can be fractionated so that only the platelets are collected for further processing in accordance with the method 50. In such instances, the platelets can be placed into a bag 10 (as described above), which is configured to transfuse the platelets back into the same or a different subject. Alternatively, a whole blood sample may not be fractionated and, instead, simply placed into a bag 10 for further processing according to the method 50.

After filling all or only a portion of the container 42 (e.g., the bag 10) with platelets or contaminated blood, all or only a portion of the container can be placed into a bag holder (such as the container 32 in FIG. 2). At Step 56, the bag holder can then be entirely or partly immersed in a cooling fluid (e.g., liquid nitrogen at about −320° F.) or a warming fluid (e.g., heated saline or water at about 90° F.). Next, the system 34 can be activated at Step 58 to impart a wrist-action motion to the container 42. The system 34 can be operated at a desired speed, rotary angle, and time sufficient to minimize the amount of RBC or platelet lysis. For example, the system 34 can be operated at a rotary angle of about 120° and at a speed of about 50 rpm for about 3 minutes. Where the method 50 entails freezing the platelets or contaminated blood, the frozen platelets or contaminated blood can be placed in a commercially available storage unit (e.g., in the vapor phase at about −310° F. just above the liquid nitrogen). Advantageously, the method 50 yields frozen platelets or contaminated blood with a significantly increased shelf life as compared to conventional methods. For instance, the shelf life of frozen blood produced by the method 50 can be greater than 2 months and, in some instances, about 10 years or more. Furthermore, any microorganisms (e.g., viruses, such as HIV or a hepatitis virus) present in the contaminated blood will be destroyed as a result of the thermal change from room temperature to freezing (e.g., −196° C.), and vice-versa. At some later date after freezing, the systems can be similarly configured (e.g., a warm water bath instead of liquid nitrogen) and operated according to the method 50 to thaw the platelets or contaminated blood.

In another aspect, it will be appreciated that contaminated blood can be exposed to a sterilizing modality either before or after completing the method 50. In one example, the sterilization modality can include UV light or pulsed light (e.g., xenon light). The contaminated blood can be exposed to the sterilization modality for a time (e.g., about 90 seconds) sufficient to neutralize any microorganisms present in the contaminated blood.

One skilled in the art will appreciate that the time periods associated with the method 50 can be varied and will depend, for example, on blood volume, container size, and thermal conductivity. Additionally, it will be appreciated that the freeze and thaw times needed for the blood to reach near thermal equilibrium with the freezing or thawing medium are those that result in maximum red cell or platelet recovery.

From the above description of the present disclosure, those skilled in the art will perceive improvements, changes and modifications. In some instances, for example, it may be important to perform the steps of the method 50 in the order described above (e.g., sequentially) to yield frozen blood with significantly increased shelf life as compared to frozen blood produced by conventional methods. Such improvements, changes, and modifications are within the skill of those in the art and are intended to be covered by the appended claims. All patents, patent applications, and publication cited herein are incorporated by reference in their entirety.

1. A bag for storing platelets or contaminated blood previously withdrawn from a subject, the bag comprising:
a flexible, closed container including an inlet therein forming part of the container, the inlet being adapted for fluid-tight connection to a tube member through which the platelets or contaminated blood are caused to flow; and
at least one removable heat transfer member adhered to an outer surface portion of the container.

2. The bag of claim 1, wherein the bag and the at least one removable heat transfer member are made of the same materials.

3. The bag of claim 1, being made of PVC with citrate, di-2-ethylhexylphthalate (DEHP) or tri-2-ethylhexyl-tri-mellitate (TEHTM) plasticizer; polyolefin (PO), poly(ethylene-co-vinyl acetate) (EVA), fluorinated polyethylene propylene (FEP), or a combination thereof.

4. The bag of claim 1, further including at least one inlet configured for fluid-tight connection to a tube member through which blood can flow.

5. The bag of claim 1, wherein the at least one heat transfer member is configured as a continuous, uninterrupted film or tape.

6. A system for freezing or thawing platelets or contaminated blood previously withdrawn from a subject, the system comprising:
an agitator drive;
a linkage arm operably connected to the agitator drive, the linkage arm configured to translate a rotary motion generated by the agitator drive to an angular reciprocation motion;
a reciprocating arm operably connected to the linkage arm; and
a container configured to hold the blood and being operably connected to a distal end of the reciprocating arm;
wherein operation of the system provides a wrist-action motion to the container and thereby minimizes the amount of red blood cell (RBC) and platelet lysis resulting from freezing or thawing of the platelets or contaminated blood.

7. The system of claim 6, wherein the agitator drive is configured to operate with a rotary angle of between about 80⁰ and about 160°.

8. The system of claim 6, wherein the agitator drive is configured to operate at a speed of about 30 rpm to about 80 rpm.

9. The system of claim 6, wherein a longitudinal axis of the reciprocating arm can extend substantially perpendicular to a longitudinal axis of the linkage arm.

10. A method for freezing or thawing platelets or contaminated blood previously withdrawn from a subject, the method comprising the steps of:
providing a system that includes an agitator drive, a linkage arm operably connected to the agitator drive, a reciprocating arm operably connected to the linkage arm, and a container operably connected to a distal end of the reciprocating arm;
filling a portion of the container with the platelets or contaminated blood;
immersing a portion of the container in a cooling or warming fluid;
activating the agitator drive to impart a wrist-action motion to the container for a time and at a speed sufficient to decrease the amount of RBC or platelet lysis resulting from freezing or thawing of the platelets or contaminated blood.

11. The method of claim 10, wherein any microorganisms present in the contaminated blood are neutralized upon freezing or thawing the contaminated blood.

12. The method of claim 11, wherein frozen blood has a shelf life of greater than about 2 months.

13. The method of claim 12, wherein the frozen blood has a shelf life of about 10 years.

14. The method of claim 11, further including exposing the contaminated blood to a sterilization modality for a period of time sufficient to neutralize any microorganisms present in the contaminated blood.

15. The method of claim 10, wherein the providing step further comprises:
providing a container comprising a bag, the bag including a flexible, closed container including an inlet therein forming part of the container, the inlet being adapted for fluid-tight connection to a tube member through which the platelets or contaminated blood are caused to flow, and at least one removable heat transfer member adhered to an outer surface portion of the container.

16. The method of claim 15, further comprising the step of adhering one or more additional heat transfer members to the bag in order to optimize heat transfer between the platelets or contaminated blood and the cooling or warming fluid.

17. The method of claim 10, wherein the activating step further includes operating the agitator drive with a rotary angle of between about 80° and about 160° and at a speed of about 30 rpm to about 80 rpm.

18. The method of claim 10, wherein each step is performed sequentially.
